(19) United States
(12) Patent Application Publication
(10) Pub. No.: US 2014/0086977 A1
(43) Pub. Date: Mar. 27, 2014

(54) TIGHT JUNCTIONS MODULATORS

(71) Applicants: Catherine Anne O'neill, Manchester (GB); Neil Kerslake Gibbs, Manchester (GB)

(72) Inventors: Catherine Anne O'neill, Manchester (GB); Neil Kerslake Gibbs, Manchester (GB)

(73) Assignee: THE UNIVERSITY OF MANCHESTER, Manchester (GB)

(21) Appl. No.: 14/008,469

(22) PCT Filed: Mar. 23, 2012
(86) PCT No.: PCT/GB2012/050655

§ 371 Date: Dec. 9, 2013

(30) Foreign Application Priority Data
Mar. 31, 2011 (GB) 1105411.1

(51) Int. Cl. A61K 31/4172; A61K 31/352

(52) U.S. Cl. CPC A61K 31/4172; A61K 31/352
USPC 424/445; 514/400; 424/443; 435/375; 514/456

(57) ABSTRACT

The present invention relates to a new therapy targeted at the restoration or improvement of tight junction function. Aberrant TJ function, is characteristic of many chronic skin diseases. The inventors demonstrated that plant polyphenols or flavonoids and Cis-urocanic acid modulates tight junction structure and function in mammalian keratinocytes and provided tight junction modulators, pharmaceutical compositions and methods for preventing or treating impaired tight junction function in stratified mammalian epithelia.

### FIELD OF THE INVENTION

The present invention relates to a new therapy targeted at the maintenance or restoration or improvement of tight junction function. Aberrant tight junction function is characteristic of many chronic skin diseases.

### BACKGROUND TO THE INVENTION

### Tight Junctions in Simple Epithelia

Epithelia are cell layers that separate compositionally distinct compartments. For example, the gut epithelium is a simple epithelium that separates the circulatory system from the contents of the gut lumen. This barrier formed by the simple gut epithelium is extremely important not only to maintain homeostasis but also to prevent penetration into the body of noxious agents and pathogens. Simple epithelial barrier function is, to a large extent, due to structures known as tight junctions (TJs). These are multiprotein complexes which seal the space between adjacent epithelial cells and prevent free movement of molecules through the paracellular space. However, to view TJs as simple occluding barriers is to underestimate their function. TJs are permeable to small and usually hydrophilic molecules and in some epithelia, the paracellular route is the main route of transport for electrolytes and water.

In simple epithelium as in the gut, disruption of TJ function usually leads to an inflammatory response and many inflammatory conditions are associated with aberrant TJ function. For example, Crohns disease, an inflammatory gut condition is associated with elevated TJ permeability. Increases in permeability lead to production of inflammatory mediators because antigens can gain access to the body. Inflammatory mediators generally further down regulate TJ function and so a vicious cycle is set in motion that is difficult to break. Although in conditions like Crohns disease, the immune response is very important, it is now generally acknowledged that therapies aimed at restoring the barrier function will be highly efficacious.

### Tight Junction Complexes

TJ complexes are composed of around 36 distinct proteins in 3 main classes:

a) transmembrane spanning proteins such as occludin and the large family of claudins
b) cytoskeletal proteins which are involved in regulating TJ permeability and
c) cytosolic plaque proteins which connect the transmembrane proteins to the actin cytoskeleton e.g. ZO-1. Although all these different proteins are important to formation and regulation, evidence suggests that it is the transmembrane proteins, particularly claudins that are the main determinants of paracellular permeability.

### Tight Junctions in Stratified Epithelia

Unlike the simple, single layered, epithelium characteristic of the gut, stratified epithelia, consisting of more than one layer exists in many mammalian body sites including skin, lips, cornea, oral cavity, esophagus, anal canal, vagina, and tongue. Although TJs have been relatively well characterised in simple epithelia like the gut, in stratified epithelium (e.g. the epidermis of skin) the importance of TJs has only recently been recognised. It was previously thought that the skin barrier function was performed solely by the uppermost layer of the epidermis, the stratum corneum. However, it is now recognised that there are two main components of skin barrier function: the stratum corneum and the TJs.

In 2002 (Furuse at al, 2002) a mouse, deficient in the major TJ protein, claudin 1, was produced. This mouse died of severe water and electrolyte loss within 24 hours of birth even though the stratum corneum (previously thought to be responsible for the skin barrier) was unperturbed in this mouse. This was the first evidence that TJs are critical to stratified epidermal barrier function. More recent data has begun to elucidate the role of TJs in epidermal barrier diseases and De Bennedetto et al (De Bennedetto et al, 2011) report on a mechanistic study suggesting that impaired TJs contribute to barrier dysfunction in atopic dermatitis subjects.

Defective TJ function is also a feature of the dry, itchy and then inflamed (xerotic) ulcer skin in venous ulcers (leg ulcers; stasis ulcers). They most commonly occur on the lower legs in up to 1.5% of the Western populations. Leg ulcers carry significant morbidity and have major socioeconomic implications. About 120,000 people are believed to be suffering from leg ulcers at any one time in the UK whilst about 500,000 people in the UK are believed to get recurrent leg ulcers. In the US leg ulcers cost $2.5 bn and 6 million lost work days annually. They are usually the result of faulty valves inside the leg veins which do not allow blood to flow up the leg towards the heart. This results in high pressure in the leg veins with consequent damage to microscopic blood vessels in the skin which becomes dry, itchy and then inflamed and xerotic and may breakdown causing an ulcer. Older people are most affected by leg ulcers. Older patients had worse loss of quality of life as did those with pain and non-healing ulcers. Interviews revealed the following effects of ulceration: pain (80.5%); itching (69.4%); altered appearance (66.7%); loss of sleep (66.6%); functional limitation (58.3%); and disappointment with treatment (50%).

Current treatments for leg ulcers may involve vascular surgery, compression stockings and topical therapies to combat the skin problems. Current topical treatments are usually moisturizers or mild steroids. These treatments are palliative and do not address the defective function and furthermore many of the topical treatments used increase the dry, itchy, inflamed, xerotic skin by inducing irritant and allergic reactions. Consequently, many ulcers fail to heal despite these treatments and become chronic causing considerable loss of quality of life to patients and an increased socioeconomic burden.

Other conditions with defective stratified epithelial barrier function associated with TJ function defects include the various forms of dermatitis, the various forms of eczema, xeroses, psoriasis, pruritus, ichthyoses, Hailey-Hailey disease, Darier's disease or Hay-Wells syndrome.

Dermatitis and eczema, (such as atopic eczema or atopic dermatitis and irritant contact dermatitis) are both characterised by a chronic phase when the skin is dry and slightly itchy with a defective epidermal harrier. The defective barrier permits the penetration of environmental triggers such as house dust mite allergens and bacterial toxins leading to a flare-up of the condition. Further the defective barrier results in water loss and drying of the skin.

Existing treatments for atopic dermatitis or eczema, irritant contact dermatitis and like skin conditions include emollient therapy and topical corticosteroids.

Emollient therapy involves a regimen consisting of emollient cream/ointments, emollient soap substitutes and bath and shower emollients. These products replace all soap and detergents and as a result produce a reduction of environmental damage to the skin barrier, however, the tight junction function is not repaired and so water loss can occur and irritants and allergens can still penetrate through the skin and trigger a flare-up.

Topical corticosteroids are used to treat a flare-up of atopic dermatitis or eczema. Adverse effects can occur in response to topical steroid treatment. Additionally, steroid paranoia can lead to non-compliance with treatment regimens.

In all these conditions, restoration or improvement of TJ function would be of great therapeutic benefit,

### SUMMARY OF THE INVENTION

There is a need to provide improved therapies to address defective tight junction (TJ) function in stratified epithelia. The present invention provides an improved therapeutic product and an improved method for the maintenance/improvement/restoration of TJ function in stratified mammalian epithelia. This is also understood as treating and/or preventing impaired TJ function. The present invention also provides an improved cosmetic product and method for improving TJ function.

A first aspect of the present invention provides a TJ modulator for use in treating or preventing impaired TJ function in stratified mammalian epithelia. The TJ modulator can be a plant polyphenol, optionally the plant polyphenol is a flavonoid and further optionally a flavonol or can be selected from quercetin, myricetin, kaempferol, quercitrin, catechin, epigallocatechin gallate, genistein, naringenin, hesperidin, apigenin, luteolin, malvidin, cyaniding, daidzein, or derivatives of quercetin, or a combination thereof. The TJ modulator can be i) cis-urocanic acid (cis-UCA), ii) ultraviolet irradiated trans-UCA, iii) cis-UCA analogue, including 2-pyrroleacrylic acid, 2-thiopheneacrylic acid, 2-furanacrylic acid, dihydrourocanic acid, 2-methylurocanic acid or 3-thiopheneacrylic acid. Alternatively, a combination of the TJ modulators are used.

In a preferred embodiment the TJ modulator comprises a flavonol for treating or preventing impaired TJ function in stratified mammalian epithelia. The flavonol may be quercetin or a quercetin derivative comprising a quercetin core structure.

In another preferred embodiment the TJ modulator comprises cis-UCA and this used in treating or preventing impaired TJ function in stratified mammalian epithelia.

The stratified mammalian epithelia includes skin and the impairment of TJ function is associated with various conditions including venous ulcers, leg ulcers, Hailey-Hailey disease, Darier's disease, Hay-Wells syndrome, dermatitis, atopic dermatitis, eczema, psoriasis, pruritus, xeroses and ichthyosis which can be treated or prevented according to the present invention.

This aspect of the invention also provides the use of a tight junction modulator in the manufacture of a medicament for treatment or prevention of impaired TJ function in stratified mammalian epithelia.

A second aspect of the present invention provides a pharmaceutical composition comprising a TJ modulator and a pharmaceutically acceptable excipient for use in treating or preventing impaired TJ function in stratified mammalian epithelia.

A third aspect of the present invention is a method of treating impaired TJ function in stratified mammalian epithelia, the method comprising administering an effective amount of a TJ modulator to a subject in need thereof.

A fourth aspect of the present invention provides an in-vitro method of improving or restoring TJ function between epithelial cells comprising administering a TJ modulator to the cells.

A fifth aspect of the present invention provides a method of improving TJ function in skin comprising topical application of a TJ modulator.

A sixth aspect of the present invention provides a preparation for improving TJ function in skin comprising one or more TJ modulators and one or more diluents or carriers.

The fifth and sixth aspects primarily concern cosmetic uses of TJ modulators and a goal is improving skin hydration.

### BRIEF DESCRIPTION OF THE DRAWINGS

FIG. 1 shows cis- and trans-UCA, i) trans-urocanic acid ii) cis-urocanic acid.

FIG. 2 shows that transepithelial electrical resistance (TEER), a marker of tight junction function, is higher in cis-UCA treated cells than untreated cells.

FIG. 3 shows that cis-UCA increases the levels of mRNA tor claudins 1, 4, occludin and ZO-1

FIG. 4 shows that cis-UCA and irradiated trans-UCA increase claudin 1 protein levels, but that unirradiated trans-UCA does not.

FIG. 5 shows that cis-UCA produces a dose dependent increase in claudin 1 protein levels.

FIG. 6 shows plant polyphenols or flavonoids useful as TJ modulators:

i) quercitrin, ii) quercetin, iii) Kaempferol, iv) catechin, v) epigallocatechin, vi) epigallocatechin gallate, vii) Myricetin, viii) genistein, ix) narigenin, x) hesperidin, apigenin, xii) luteolin, xiii) malvidin, xiv) cyanidin, xv) daidzein.

FIG. 7 shows the effects of flavonoids on TEER in human primary keratinocytes when treated with a) Myricetin, b) quercetin and c) Kaempferol.

FIG. 8 shows the effects of differing doses of quercetin on TEER in human primary keratinocytes.

### SPECIFIC DESCRIPTION

Stratified epithelium of various anatomical sites (e.g. skin, lips, cornea, oral cavity, oesophagus, anal canal, vagina and tongue) acts as a major barrier of the body against environmental insult and retains moisture. In the skin, flattened keratinocytes (squames) form the outermost layer of the epidermis, the stratum corneum. Defective skin barrier function results in increased water loss and increased ingress of microorganisms, viruses, allergens, toxins, irritants and other noxious exogenous factors. As noted above, existing treatments for skin conditions concerning impaired skin barrier function focussed on the stratum corneum. More recently, it has been recognised that TJs also have an important role to play in maintaining the skin barrier, and that defective TJs, leading to impaired TJ function, are the focus of further research to develop additional and improved therapies.

The present inventors have studied the role of UCA, its analogues, and other TJ modulating compounds on TJ structure and function.

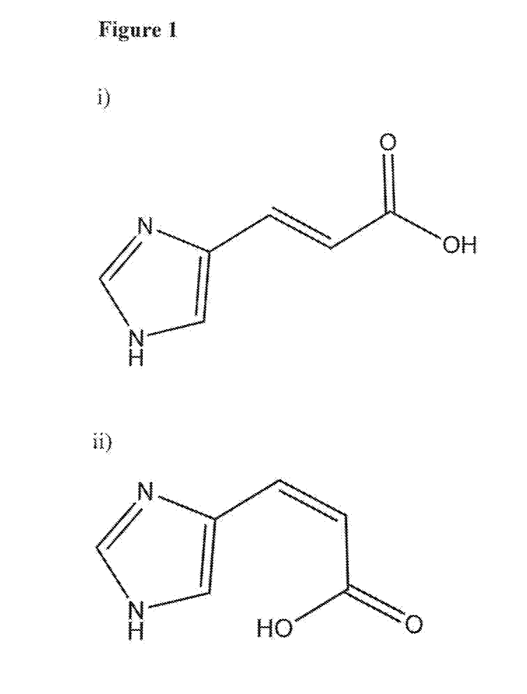

UCA is formed in the upper layers of the epidermis where it is synthesised as the trans-isomer (trans-UCA) (FIG. 1) in a deamination reaction, catalysed by histidase (histidine ammonia-lyase). Trans-UCA accumulates in the skin to high concentration (approximately 20 nmol cm⁻²)

On absorption of ultraviolet radiation (UVR) trans-UCA is photoisomerised to the cis-UCA isomer (FIG. 1). This reaction is dose-dependent until a photostationary state is reached at which point the cis-UCA concentration is approximately 60-70%. The UVR dose required to produce maximal cis-UCA acid is less than the dose required to produce sunburn. The exact function of cis-UCA acid in human skin is unclear although there is evidence to suggest that it acts as a ‘natural sunscreen’ against UVR-induced skin damage and that it may alter immune reactions after UVR exposure.

It should be noted that cis-UCA is the photoproduct formed from trans-UCA mammalian skin after UV exposure. However, that it is also known that UV exposure decreases mammalian TJ function. It is therefore counterintuitive that a photoproduct formed in the skin during UV irradiation should possess the ability to increase TJ function. Nevertheless, when exploring the properties of trans and cis-UCA the present inventors demonstrated that cis-UCA had an isomer specific effect of upregulating TJ function in cultured primary human keratinocytes.

Therefore the present invention relates to cis-UCA in preventing and treating impaired TJ function and in improving or maintaining TJ function in stratified mammalian epithelia. Derivatives of cis-UCA may also be used. Irradiated trans-UCA, containing cis-UCA is also embraced by this invention, as are cis-UCA analogues including 2-pyrroleacrylic acid, 2-thiopheneacrylic acid, 2-furanacrylic acid, dihydrourocanic acid, 2-methylurocanic acid and 3-thiopheneacrylic acid and derivatives thereof. The cis-UCA analogues are structurally similar to cis-UCA and therefore have similar beneficial effects on tight junctions. In certain embodiments combinations comprising one or more of cis-UCA, irradiated trans-UCA and cis-UCA analogues may be used.

Furthermore, the plant polyphenol compounds quercetin, quercetrin, kaempferol, catechin, epigallocatechin gallate, myricetin or genistein are known to up regulate TJ function in simple epithelia of the gut and the present inventors show that they too have beneficial effects on the TJs of stratified epithelia. Further plant polyphenol compounds in the same family and therefore beneficial for TJs are naringenin, hesperidin, apigenin, luteolin, cyanidin and daidzein.

Plant polyphenols of importance in the present invention include the class of compounds known as the flavonoids (or bioflavonoids). The flavonoids are polyphenolic compounds which may be visualized as a core structure of 15 carbon atoms; two benzene rings joined by a short linear three carbon chain. One of the carbons of the short chain is always connected to a carbon of one of the benzene rings, either directly or through an oxygen bridge thereby forming a third middle ring, which may be 5- or 6-membered. Flavonoids generally have a flavone backbone (2-phenyl-1,4-benzopyrone). Flavonoids are the most common group of polyphenolic compounds in the human diet and are found ubiquitously in plants. Good dietary sources of flavonoids include all citrus fruits, berries, ginko biloba, onions (particularly red onion), parsley, pulses, tea (especially white and green tea), red wine, seabuckthorn and dark chocolate (with a cocoa content of seventy percent or greater). Together with carotenes, flavonoids are often responsible for the colouring of fruits, vegetables and herbs. Classes of compounds falling under the term flavonoids include flavones, flavonols, flavanones, flavanonols, isoflavones and anthocyanidin. Such compounds have been used in cosmetics for their pleasant scents and for their antioxidant properties.

In an embodiment of the present invention the flavonoids used in preventing and treating impaired function and in improving or maintaining TJ function in stratified mammalian epithelia are from the flavonol class. Flavonols (with an “o”) are a class of flavonoids that have the 3-hydroxyflavone backbone (IUPAC name: 3-hydroxy-2-phenylehomen-4-one). Their diversity can stem from the different positions of the phenolic-OH groups. In particular the flavonols quercetin, myricetin, Kaempferol, quercitrin and derivatives of quercetin (compounds having quercetin as the core structure) may be used in the present invention. As described below, the inventors have demonstrated that these plant polyphenols are able to improve function and enhance the skin barrier.

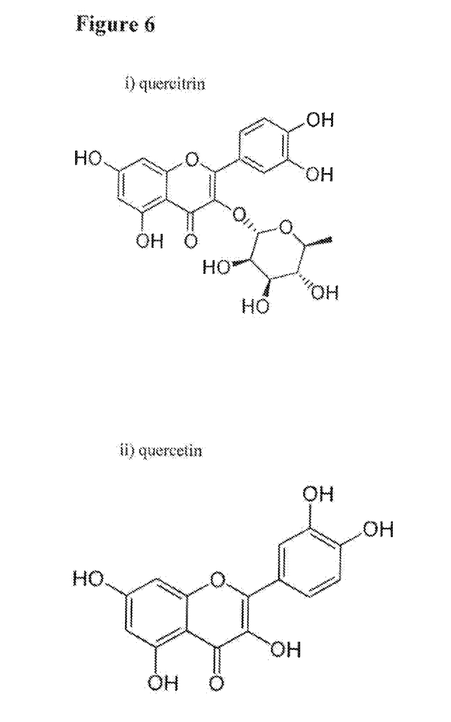

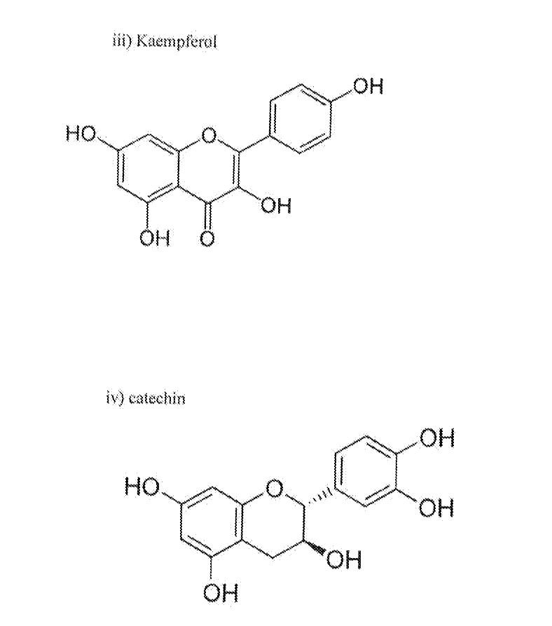

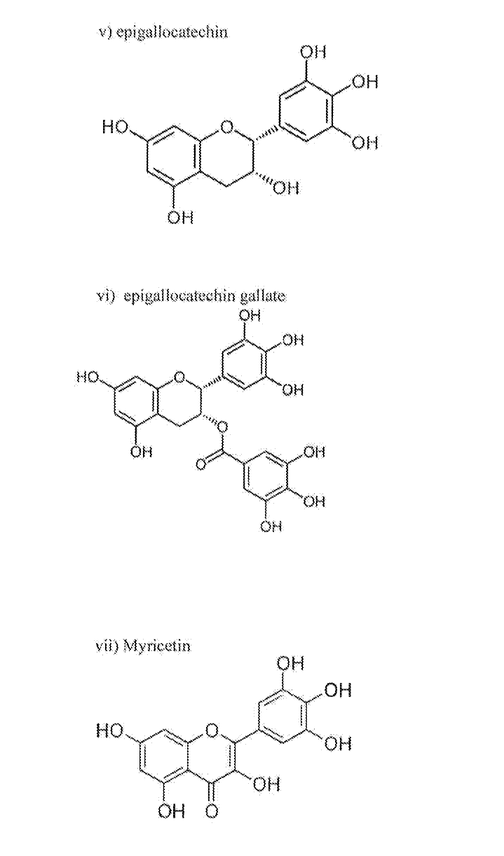

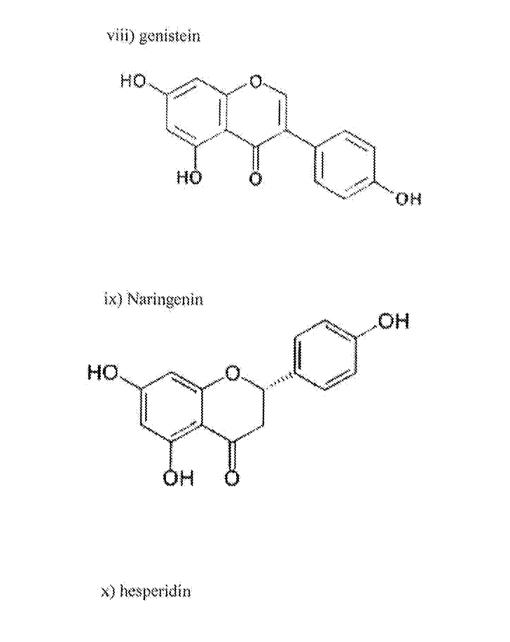

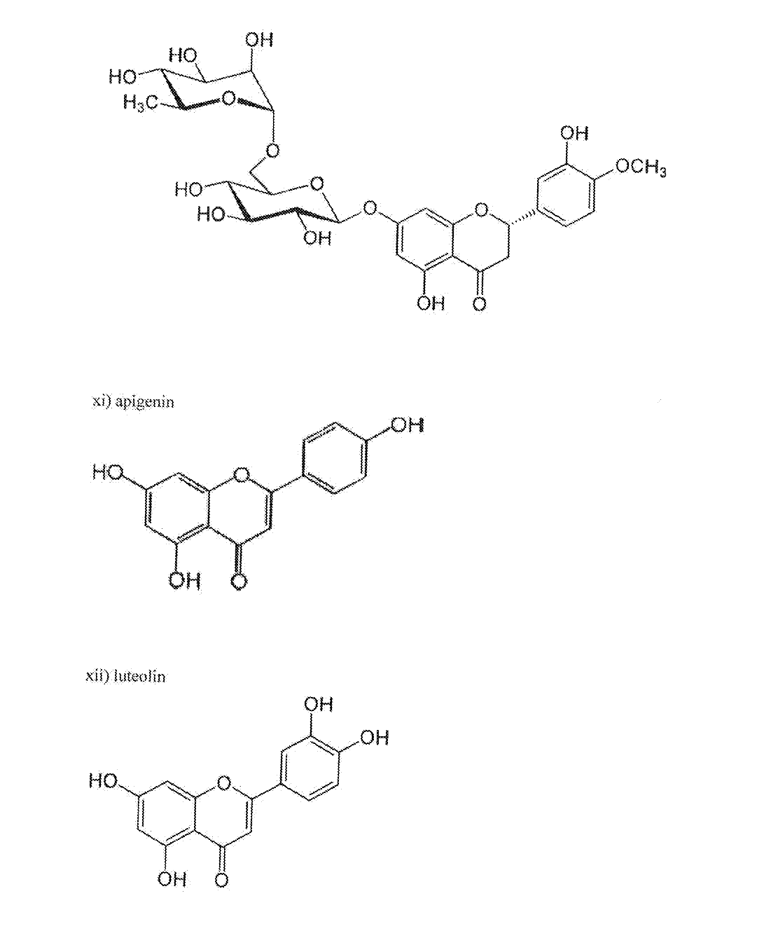

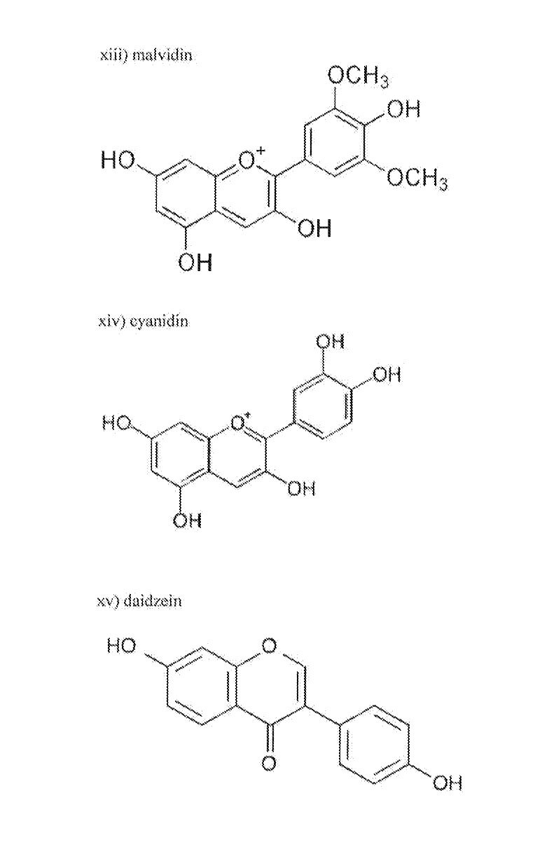

FIG. 6 shows plant polyphenols or flavonoids of use in the present invention including i) quercitrin, ii) quercetin, iii) kaempferol, iv) catechin, v) epigallocatechin, vi) epigallocatechin gallate, vii) myricetin, viii) genistein, ix) narigenin, x) hesperidin, xi) apigenin, xii) luteolin, xiii) malvidin, xiv) cyanidin, xv) daidzein.

In certain embodiments derivatives of flavonols, including derivatives of quercetin, myricetin kaempferol, quercitrin, catechin, epigallocatechin, epigallocatechin gallate, genistein, narigenin, hesperidin, apigenin, luteolin, malvidin, cyanidin, daidzein may be used in preventing and treating impaired TJ function and in improving or maintaining TJ function in stratified mammalian epithelia. Derivatives include substitution of one or more hydroxyl group with a C1-C6 lower alkyl group, such as a methyl, ethyl, propyl or butyl group, preferably a methyl group. Derivatives of quercetin are preferred. It is believed that quercetin is the pharmacophore and compounds retaining a quercetin core structure are valuable as TJ modulators.

In certain embodiments a combination comprising more than one plant polyphenol or flavonoid may be used to prevent or treat impaired TJ function or to improve or maintain TJ function in stratified mammalian epithelia. For example two, three or more plant polyphenols or flavonoids may be used. Therefore compositions of the present invention may comprise one, two, three or more plant polyphenols or flavonoids.

In a further embodiment one or more plant polyphenols or flavonoids may be used in combination with one or more of cis-UCA, irradiated trans-UCA, or cis-UCA analogue(s) in preventing or treating impaired TJ function or in improving or maintaining TJ function in stratified mammalian epithelia.

The barrier function of stratified mammalian epithelia is reduced if that epithelia has impaired TJ function. The skin, lips, cornea, oral cavity, esophagus, anal canal, vagina, and tongue of mammals all have stratified epithelia containing TJs, the function of which can be improved, maintained or restored by the TJ modulators, pharmaceutical compositions and methods of the present invention.

A condition treated or prevented by the present invention is impaired TJ function. Further conditions treated by the present invention include venous ulcer, leg ulcer, Hailey-Hailey disease, Darier's disease, Hay-Wells syndrome, dermatitis, atopic dermatitis, psoriasis, ichthyosis and xeroses.

In certain embodiments the TJ modulators, methods, compositions and preparations of the present invention comprising TJ modulators may be used in treating or preventing the various forms of dermatitis, the various forms of eczema, the xeroses, psoriasis, pruritus and the ichthyoses, venous ulcer, leg ulcer, Hailey-Hailey disease, Darier's disease or Hay-Wells syndrome. The forms of dermatitis which may be treated, prevented or improved by a TJ modulator of the present invention include atopic dermatitis, contact dermatitis, irritant contact dermatitis, seborrhoeic dermatitis or non-atopic dermatitis.

The TJ modulators, pharmaceutical compositions and methods of the present invention can be used in medicine to treat human patients and can be used in veterinary medicine to treat other mammals such as a horse, dog, eat, cow, sheep, pig or goat.

The TJ modulators, preparations and methods of the present invention can be used for cosmetic purposes. Improving TJ function in skin improves skin hydration and improves skin moisturization, tone and texture.

Compositions and preparations of the present invention can be pharmaceutical or cosmetic. Generally the compositions and preparations of the present invention are formulated with dermatologically acceptable carrier(s), vehicle(s) and excipient(s). These compositions and preparations can be for topical application. A preferred embodiment involves topical application to an affected area. Compositions and preparations of the present invention can be in the form of lotions, emulsions, creams, sprays, gels, hydrogels, powders, ointments, pastes, suppositories, foams, aerosols, wipes, impregnated dressings or impregnated garments. In topical dermatological compositions and preparations of the present invention a TJ modulator may be formulated with suitable agents such as humectants, emollients, gelling and thickening agents preservatives, penetration enhancers and optionally fragrances and other carriers, vehicles or excipients. A preferred topical treatment for ulcers is a garment, such as socks or tights, from which a composition comprising a TJ modulator is released.

Alternative routes of administration for the TJ modulators of the present invention include injection and oral administration. In oral dosage forms one or more TJ modulator may be combined with orally acceptable carrier(s), vehicles or excipient(s). For example the oral formulation may be as a pill, tablet, pustule, drink, food supplement or capsule.

In this disclosure concentrations of a TJ modulator and other components of the compositions and preparations of the present invention may be expressed as % w/v (percentage weight/volume) in which a solution with 1 g of a TJ modulator or other component in 100 ml of solution may be referred to as 1% or 1% w/V.

Compositions and preparations of the present invention for topical application to stratified mammalian epithelia may comprise at least 0.002% w/v, at least 0.003% w/v at least 0.005% w/v, at least 0.01% w/v, at least 0.02% w/v, at least 0.03% w/v, at least 0.05% w/v, at least 0.1% w/v, at least 0.2% w/v, at least 0.3% w/v or at least 0.5% w/v of a TJ modulator.

Compositions and preparations of the present invention for topical application to stratified mammalian epithelia may comprise up to 1% w/v, 1.5% w/v, 2% w/v, 2.5% w/v, 3% w/v, 3.5% w/v, 4% w/v, 4.5% w/v, 5% w/v, 6% w/v, 7% w/v, 8% w/v, 9% w/v or 10% w/v of a TJ modulator.

Typical pharmaceutical compositions may comprise a TJ modulator from about 0.03% w/v to about 5% w/v, optionally from about 0.05% w/v to about 2% w/v.

Typical cosmetic preparations may comprise a TJ modulator from about 0.003% w/v to 2% w/v optionally from about 0.005% w/v to about 1% w/v.

Plant polyphenols or flavonoids may be present in compositions and preparations of the present invention at least 10 μM, at least 50 μM, at least 100 μM, at least 200 μM, at least 500 μM or at least 1 mM.

Plant polyphenols or flavonoids may be present in compositions and preparations of the present invention at up to 10 mM, up to 50 mM, 66 mM (where 66 mM of quercetin is approximately 2% w/v), 75 mM, 100 mM, 200 mM or 300 mM.

Oral dosage forms of the present invention may comprise a TJ modulator in an amount of at least 20 mg, at least 30 mg or at least 50 mg. Such oral dosage forms may comprise a TJ modulator in amounts up to 500 mg, 1 g, 5 g, 10 g, 50 g or 100 g. One, two, three, four or more oral dosage forms may be administered a day.

Transepithelial electrical resistance (TEER) measurements are used in epithelial biology as a marker of TJ integrity. Electrical resistance is a measure of how easily ions can pass through a particular transport route. The higher the resistance the harder it is for ions to pass. Whilst the TEER of a cell monolayer is a function of both transcellular and paracellular transport routes, the value of TEER is, however, dominated by the contribution from the paracellular route. TEER of monolayers can be measured using an Epithelial Voltmeter (for example EVOM from WPI Ltd). This instrument passes a current across a monolayer of cells and calculates the resistance according to Ohm's law (V=IR, where V is potential difference, I is current and R is resistance). Therefore increased TEER means unregulated TJ function. Preferably TEER measurements are carried out at room temperature.

TEER measurements can be used as an indicator of improving TJ function or an indicator of treating impaired TJ function according to the present invention. Furthermore the effect of TJ modulators on TJ function may be assayed using TEER measurements. It will be understood that increased TEER measurements correlates with improved TJ function. Upregulating or improving TJ function leads to improvements in skin diseases.

Transepidermal water loss (TEWL) is used as a measurement of water loss via the skin and TEWL is therefore a measure of the integrity of the skin barrier. A greater water loss via the skin indicates a defective skin barrier. The skin barrier function is now understood to be provided by a combination of the stratum corneum and the TJs. However, as described above, it has been shown that defective TJs in vivo leads to massive water loss, even if the stratum corneum is unperturbed. Therefore improving TJ function in a mammal reduces water loss via the skin and this may be measured by TEWL, a lower TEWL measurement correlating with improving TJ function.

TEWL measurements can be used as an indicator of improving TJ function or an indicator of treating impaired TJ function according to the present invention. Furthermore the effect of TJ modulators on impaired TJ function in vivo may be assayed using TEWL measurements. Upregulating or improving TJ function leads to improvements in skin diseases. Alternatively the effect of TJ modulators on impaired TJ function may be assayed by questioning subjects having impaired TJ function and to whom a TJ modulator has been administered. Various questioning protocols are available.

### EXAMPLES

### Experiment 1

### Cis-UCA Augments TJ Function in NHEK

Normal primary human keratinocytes (NHEK) were grown on Thincerts™ in medium containing 0.6 mM calcium until the cells were confluent. TJ formation was the induced by calcium switch and 25 ug/ml cis-UCA was added to the cells which were incubated for 96 hours and the transepithelial electrical resistance (TEER) measured every 24 hours post calcium switch.

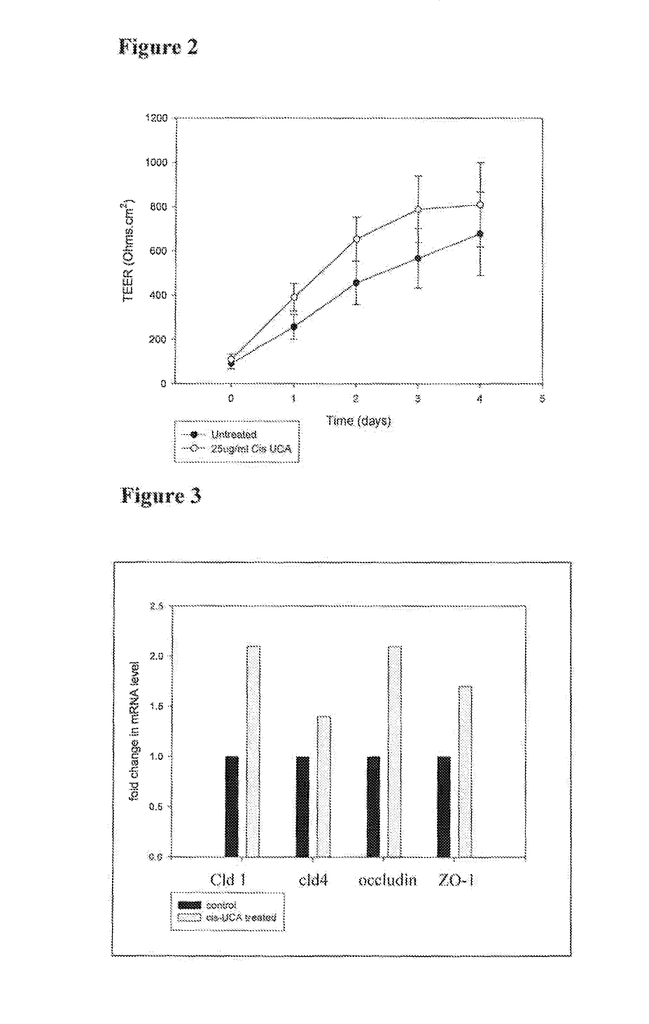

In cis-UCA treated cells, TEER was approximately 150 ohms.cm² higher than in untreated cells (FIG. 2),

### Experiment 2

### Cis-UCA Increases the Expression of TJ Protein mRNA's in Keratinocytes

Normal primary human keratinocytes (NHEK) were grown in proprietary medium containing 0.6 mM calcium chloride. When the cells reached confluence they were placed in medium containing 10.8 mM calcium chloride (calcium switch) and 25 ug/ml cis-UCA was added. Cells were incubated for 48 hours and message levels for the TJ genes claudins 1 and 4, occludin and ZO-1 were quantified using real-time PCR using non-treated keratinocytes as a control (FIG. 3).

### Experiment 3

### Cis but not Trans UCA Increases Claudin 1 Expression in Keratinocytes

Normal primary human keratinocytes (NHEK) were grown in proprietary medium containing 0.6 mM calcium chloride. When the cells reached confluence they were placed in medium containing 1.8 mM calcium chloride (calcium switch) and either cis-UCA, irradiated trans-UCA, unirradiated trans-UCA or control medium. Cells were the incubated for 96 hours during which time TJs form. At 24, 48, 72 or 96 hours post treatment, cells were harvested and claudin 1 levels analysed by immunoblot.

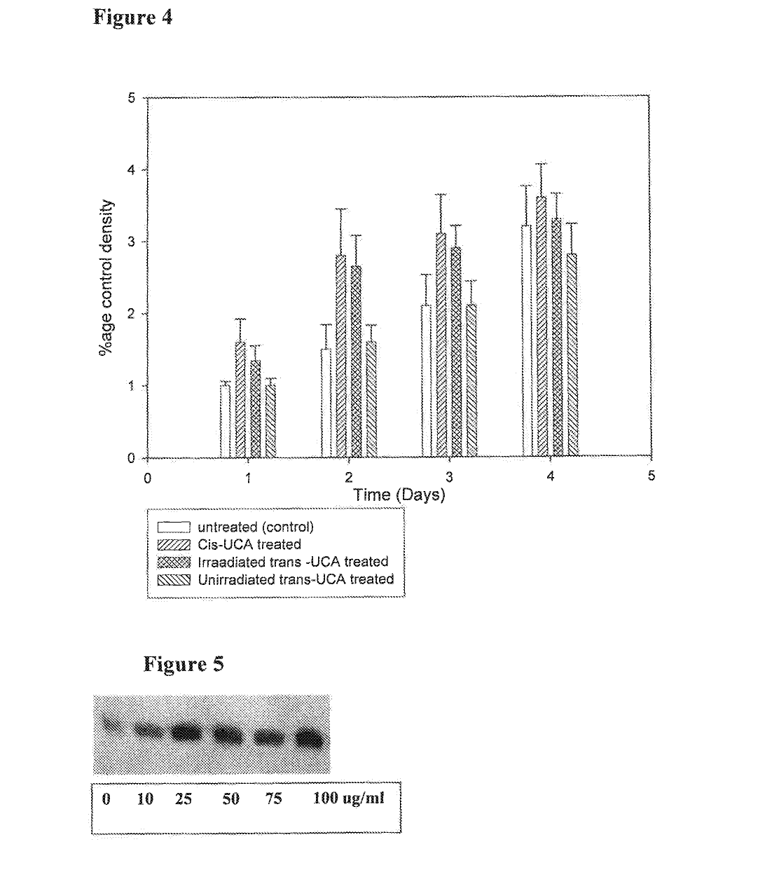

Cis- or irradiated trans-UCA produced a time dependent increase in claudin 1 levels (FIG. 4). However, unirradiated trans-UCA had no effect on claudin 1 levels in NHEK (FIG. 4).

### Experiment 4

### Cis Urocanic Acid Increases Claudin 1 Expression in a Dose Dependent Manner

NHEK (growing as described above) were treated with different concentrations of cis-UCA for 48 hours after which claudin 1 levels were examined using immunoblotting. Cis-UCA produced a dose dependent increase in claudin 1 levels which was greatest when cells were treated with 25 ug/ml cis-UCA (FIG. 5). Increasing the amount of cis-UCA did not further increase claudin 1 levels.

### Experiment 5

### Preparation of Topical Formulations of Cis-UCA

The following topical creams were prepared.

Topical 4% w/w cis-UCA Cream

| Ingredient | % w/w |
|---|---|
| Cis-urocanic acid | 4.0 |
| Emusifying Ointment BP | 30.0 |
| Isopropyl Myristate | 5.0 |
| Hydroxyethylcellulose | 0.2 |
| Glycerol | 1.0 |
| Phenoxyethanol | 1.0 |
| Purified Water | 58.8 |
| TOTAL | 100 |

Topical 1% w/w cis-UCA Cream

| Ingredient | % w/w |
|---|---|
| Cis-urocanic acid | 1.0 |
| Emusifying Ointment BP | 30.0 |
| Isopropyl Myristate | 5.0 |
| Hydroxyethylcellulose | 0.2 |
| Glycerol | 1.0 |
| Phenoxyethanol | 1.0 |
| Purified Water | 61.8 |
| TOTAL | 100 |

Topical 2% w/w cis-UCA Gel

| Ingredient | % w/w |
|---|---|
| Cis-UCA | 2.0 |
| Hydroxyethylcellulose | 1.8 |
| Propylene Glycerol | 20.0 |
| Phenoxyethanol | 1.0 |
| Purified Water | 61.8 |
| Purified Water | 75.2 |

### Further Explanation

### Cis-Urocanic Acid Modulates Claudin-1 Expression and Tight Junction Function in Human Keratinocytes

Exposure of human skin to solar simulated ultraviolet radiation (UVR) transiently impairs skin barrier function (SBF) which recovers over a period of 2-14 days. Recent evidence suggests that epidermal tight junctions (TJ) in the granular layer are critically involved in maintaining SBF with mice lacking the TJ protein, claudin-1 (Cld-1) dying of dehydration within 24 h of birth. Trans-urocanic acid (trans-UCA) is a major epidermal chromophore located in the stratum corneum and exposure of human skin to UVR photoisomerises trans-UCA to cis-UCA. In a previous Affymetrix study, the Cld-1 encoding gene, CLDN1, was listed as one of several markers induced by cis- but not trans-UCA, in normal human epidermal keratinocytes (NHEK) (Kaneko et al, J Immunol 2008, 181, 217-24). We therefore examined UCA isomer-dependent changes in Cld-1 protein expression (assessed by immunoblotting) and TJ function (assessed as transepithelial electrical resistance; TEER) in differentiated NHEK in vitro.

Cis-UCA produced a time and concentration dependent increase in Cld-1 protein which was maximal 48 h post-treatment with 25 μg/ml (increased ˜3 fold; n=4, p=0.02), in cis-UCA treated cells, TEER was ˜150 ohms/cm² higher than in untreated cells (n=6, p=0.004). Similar effects were observed after treatment of differentiated NHEK with UVR-irradiated trans-UCA (comprising ˜50% cis-UCA). In contrast, trans-UCA had no effect on Cld-1 expression or TEER.

This study demonstrates that cis-UCA upregulates Cld-1 protein levels and TJ function in differentiated NHEK in an isomer-specific manner. Our data suggest that production of cis-UCA from endogenous trans-UCA may have an important role in the recovery of human SBF following exposure to UVR and that exogenous cis-UCA may offer a therapeutic approach in skin conditions associated with a compromised SBF.

### Example 6

### Plant Polyphenols Augment TJ Function in NHEK

Normal human epidermal keratinocytes (NHEK) were purchased from Promocell (Heidelberg, Germany) and grown on Thincerts™ in proprietary medium containing 0.06 mM calcium chloride. ‘Thincerts’ are small chambers containing a filter on which the cells are grown. The Thincert™ is housed within a conventional 12 well tissue culture plate. This arrangement allows treatment of the cells from either the basal or apical side.

When the cells were confluent, the medium was replaced with new medium but containing 1.8 mM calcium chloride. This induces tight junction formation in NHEK. In experiments where plant polyphenols were tested, a known volume of the plant polyphenol (dissolved in vehicle at a known concentration) was added to both apical and basal chambers of the Thincert™ at the same time as the medium was replaced. In this experiment the plant polyphenols quercetin, Myricetin and Kaempferol were tested.

Tight junctions form over 4 days and this was monitored by measuring the transepithelial electrical resistance (TEER) using an EVOM (world precision instruments). TEER was measured every 24 hours post addition of the test polyphenols and the TEER of control untreated cell monolayers was compared to that of treated cell monolayers. The results were analysed by Mann Whitney U test and TEER was deemed to be significantly different between treated and control where the p value was less than 0.05.

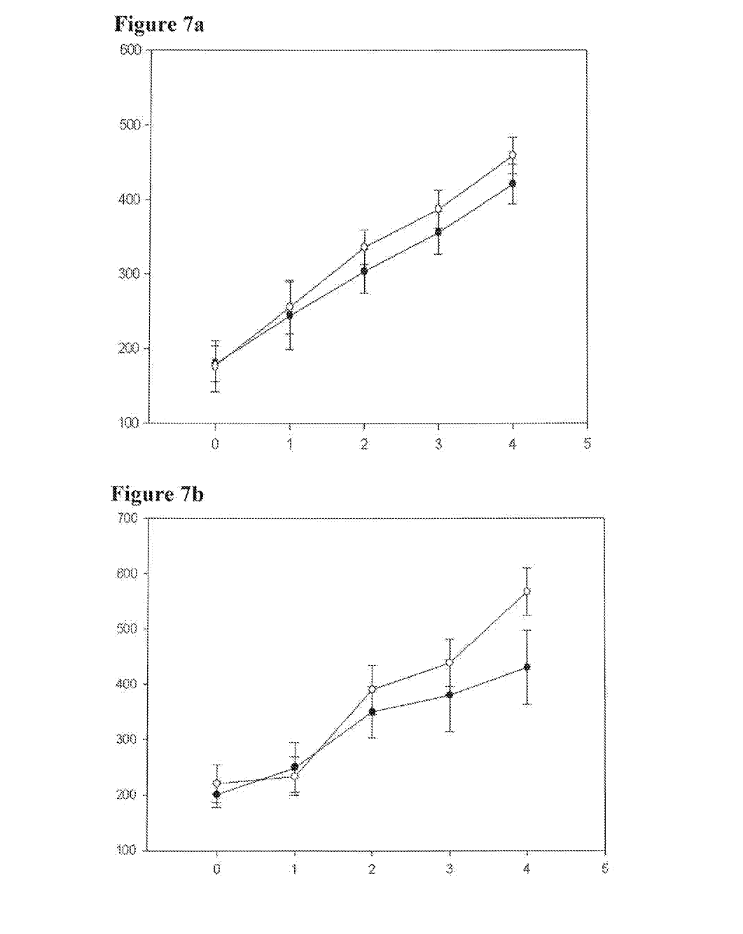

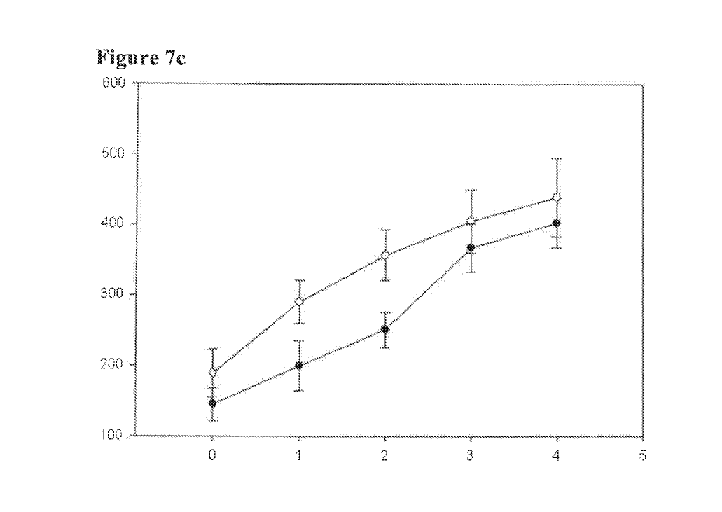

As shown in FIG. 7a and FIG. 7b myricetin and quercetin respectively show statistically significant differences between the control and the treated cells at day four, in FIGS. 7a, 7b and 7c the x-axis shows time (days) and the y-axis shows TEER (Ohms.cm²). FIG. 7a shows that myricetin increases TEER in human primary keratinocytes. The open circles illustrate results for cells treated with 100 μM myricetin, whilst the filled circles illustrate results for untreated control cells. FIG. 7b shows that quercetin increases TEER in human primary keratinocytes. The open circles illustrate results for cells treated with quercetin, whilst the filled circles illustrate results for untreated control cells. Therefore, both quercetin and Myricetin significantly increase TEER in human primary keratinocytes.

As shown in FIG. 7c, Kaempferol increases TEER in human primary keratinocytes at day four and reduces the time to barrier formation in human primary keratinocytes. The open circles illustrate results for cells treated with 100 μM Kaempferol, whilst the filled circles illustrate results for untreated cells.

### Example 7

### Quercetin has Dose Dependent Effects on TEER in Human Primary Keratinocytes

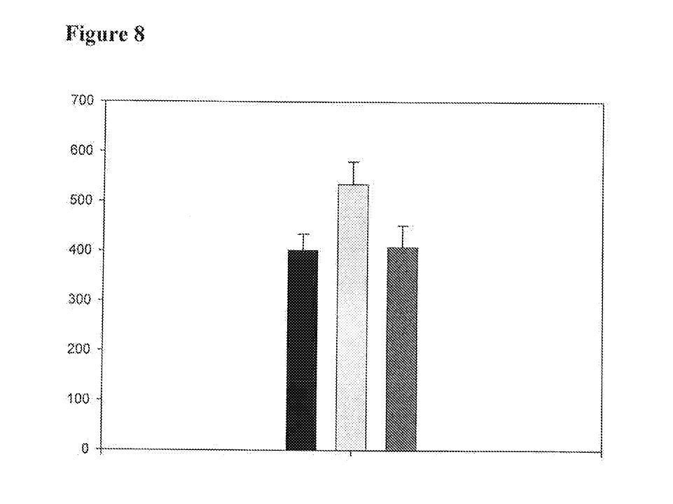

The methodology set out in Example 6 was repeated, using quercetin at different concentrations. The results are shown in FIG. 8 in which the first bar represents the results for control—untreated cells, the second bar represents the results for cells treated with 100 μM quercetin and the third bar represents the results for cells treated with 10 μM quercetin (see key below). The y-axis represents TEER at 4 days (Ohms.cm²). The results shown in FIG. 8 confirm that higher quantities of quercetin have a more beneficial effect on TJ formation and on increasing TEER.

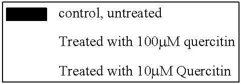

### Example 8

### Preparation of Topical Formulations of Plant Polyphenols

The following topical creams were prepared

Topical 0.003% quercetin formulation

| Ingredient | % w/v |
|---|---|
| Quercetin | 0.003% |
| Emulsifying Ointment BP ™ | 30.0 |
| (White Soft Paraffin, Liquid | |
| Paraffin, Emusifying Wax | |
| mixture) | |
| Isopropyl Myristate | 5.0 |
| Hydroxyethylcellulose | 0.2 |
| Glycerol | 1.0 |
| Phenoxyethanol | 1.0 |
| Purified Water | 62.797 |

Topical 0.03% quercetin formulation

| Ingredient | % weight/volume |
|---|---|
| Quercetin | 0.03 |
| Emusifying Ointment BP ™ | 30.0 |
| (White Soft Paraffin, Liquid | |
| Paraffin, Emusifying Wax | |
| mixture) | |
| Isopropyl Myristate | 5.0 |
| Hydroxyethylcellulose | 0.2 |
| Glycerol | 1.0 |
| Phenoxyethanol | 1.0 |
| water | 62.77 |

Topical 0.5% quercetin formulation

| Ingredient | % weight/volume |
|---|---|
| Quercetin | 0.5 |
| Emusifying Ointment BP ™ | 30.0 |
| (White Soft Paraffin, Liquid | |
| Paraffin, Emusifying Wax | |
| mixture) | |
| Isopropyl Myristate | 5.0 |
| Hydroxyethylcellulose | 0.2 |
| Glycerol | 1.0 |
| Phenoxyethanol | 1.0 |
| water | 62.30 |

Topical 2% quercetin formulation

| Ingredient | % weight/volume |
|---|---|
| Quercetin | 2.0 |
| Emusifying Ointment BP ™ | 30.0 |
| (White Soft Paraffin, Liquid | |
| Paraffin, Emusifying Wax | |
| mixture) | |
| Isopropyl Myristate | 5.0 |
| Hydroxyethylcellulose | 0.2 |
| Glycerol | 1.0 |
| Phenoxyethanol | 1.0 |
| water | 60.8 |

Topical 0.03% myricetin formulation

| Ingredient | % weight/volume |
|---|---|
| Myricetin | 0.03 |
| Emusifying Ointment BP ™ | 30.0 |
| (White Soft Paraffin, Liquid | |
| Paraffin, Emusifying Wax | |
| mixture) | |
| Isopropyl Myristate | 5.0 |
| Hydroxyethylcellulose | 0.2 |
| Glycerol | 1.0 |
| Phenoxyethanol | 1.0 |
| water | 62.77 |

Topical 0.03% kaempferol formulation

| Ingredient | % weight/volume |
|---|---|
| Kaempferol | 0.03 |
| Emusifying Ointment BP ™ | 30.0 |
| (White Soft Paraffin, Liquid | |
| Paraffin, Emusifying Wax | |
| mixture) | |
| Isopropyl Myristate | 5.0 |
| Hydroxyethylcellulose | 0.2 |
| Glycerol | 1.0 |
| Phenoxyethanol | 1.0 |
| water | 62.77 |

### Example 9

### Preparation of Oral Formulations of Plant Polyphenols

| Flavonoid | 10-50 | mg |
|---|---|---|
| Lactosum Ph. Eur. | 67.8 | mg |
| Cellulose, microcryst. (Avicel) | 31.4 | mg |
| Amberlite ® IRP88* | 1.0 | mg |
| Magnesii stearas Ph. Eur. | quantum sufficiat | |
| Coating: | | |
| Hydroxypropyl methylcellulose | about 9 | mg |
| Mywacett ® 9-40 T** | about 0.9 | mg |

### REFERENCES

Furuse M. et al J. Cell Biol 2002; 156: 1099-111
Kaneko K, et al J. Immunol. 2008, 181; 217-224
De Denedetto A. et al J. Allergy Clin. Immunol. 2011; 127: 773-86

1-10. (canceled)

11. A pharmaceutical composition comprising a tight junction modulator and a pharmaceutically acceptable carrier, vehicle or excipient.

12. The pharmaceutical composition according to claim 11, wherein the tight junction modulator is a plant polyphenol, optionally the plant polyphenol is a flavonoid and further optionally a flavonol.

13. The pharmaceutical composition according to claim 12, wherein the plant polyphenol is selected from hesperidin, quercetin, myricetin, kaempferol, quercitrin, catechin, epigallocatechin gallate, genistein, maringenin, apigenin, luteolin, malvidin, cyanidin, daidzein, and a derivative of quercetin, or a combination thereof.

14. The pharmaceutical composition according to claim 11, wherein the tight junction modulator is
i) cis-urocanic acid (cis-UCA), or
ii) ultraviolet irradiated trans-UCA, or
iii) a cis-UCA analogue, optionally selected from 2-pyrroleacrylic acid, 2-thiopheneacrylic acid, 2-furanacrylic acid, dihydrourocanic acid, 2-methylurocanic acid or 3-thiopheneacrylic acid,
or a combination thereof.

15. The pharmaceutical composition according to claim 11, wherein the composition is for topical administration and is optionally in the form of a lotion, emulsion, cream, spray, gel, hydrogel, ointment, paste, suppository, powder, foam, aerosol, wipe, impregnated dressing or impregnated garment.

16. (canceled)

17. (canceled)

18. A method of treating impaired tight junction function in stratified mammalian epithelia, the method comprising administering an effective amount of a tight junction modulator to a mammalian subject in need thereof.

19. The method according to claim 18, wherein the stratified mammalian epithelia is selected from the group consisting of skin, lips, cornea, oral cavity, esophagus, anal canal, vagina, and tongue.

20. The method according to claim 18, wherein the subject has a condition selected from the group consisting of dermatitis, eczema, xeroses, psoriasis, pruritus, ichthyoses, venous ulcer, leg ulcer, Hailey-Hailey disease, Darier's disease and Hay-Wells syndrome.

21. The method according to claim 20, wherein the dermatitis is selected from the group consisting of atopic dermatitis, contact dermatitis, irritant contact dermatitis, seborrhoeic dermatitis and non-atopic dermatitis.

22. The method according to claim 18, wherein the mammalian subject is selected from the group consisting of human, horse, dog, cat, cow, sheep, pig and goat.

23. The method according to claim 18, wherein the tight junction modulator is a plant polyphenol.

24. The method according to claim 18, wherein the tight junction modulator is a flavonoid and optionally is a flavonol.

25. The method according to claim 18, wherein the tight junction modulator is selected from the group consisting of hesperidin, quercetin, myrketin, kaempferol, quercitrin, catechin, epigallocatechin gallate, genistein, maringenin, apigenin, luteolin, malvidin, cyanidin, daidzein, and a derivative of quercetin, or a combination thereof.

26. The method according to claim 18, wherein the tight junction modulator is selected from the group consisting of
i) cis-urocanic acid (cis-UCA),
ii) ultraviolet irradiated trans-UCA, and
iii) a cis-UCA analogue selected from the group consisting of 2-pyrroleacrylic acid, 2-thiopheneacrylic acid, 2-furanacrylic acid, dihydrourocanic acid, 2-methylurocanic acid and 3-thiopheneacrylic acid,
or a combination thereof.

27. The method according to claim 18, wherein the method comprises topical administration to an affected area of the subject.

28. An in-vitro method of improving or restoring tight junction function between epithelial cells comprising administering a tight junction modulator to the cells.

29. The in-vitro method of improving or restoring tight junction function according to claim 28, wherein the tight junction modulator is a plant polyphenol, optionally the plant polyphenol is a flavonoid and further optionally a flavonol.

30. The in-vitro method of improving or restoring tight junction function according to claim 29, wherein the plant polyphenol is selected from hesperidin, quercetin, myricetin, kaempferol, quercitrin, catechin, epigallocatechin gallate, genistein, maringenin, apigenin, luteolin, malvidin, cyanidin, daidzein, and a derivative of quercetin or a combination thereof.

31. The in-vitro method of improving or restoring tight junction function according to claim 28, wherein the tight junction modulator is selected from
i) cis-urocanic acid (cis-UCA), or
ii) ultraviolet irradiated trans-UCA, or
iii) a cis-UCA analogue, optionally selected from 2-pyrroleacrylic acid, 2-thiopheneacrylic acid, 2-furanacrylic acid, dihydrourocanic acid, 2-methylurocanic acid and 3-thiopheneacrylic acid,
or a combination thereof.

32. The in-vitro method of improving or restoring tight junction function according to claim 28, wherein the cells are keratinocytes.

33. A method of improving tight junction function in skin comprising topical application of a tight junction modulator.

34. The method according to claim 33, wherein the method is for improving skin hydration.

35. The method according to claim 33, wherein the tight junction modulator is a plant polyphenol, optionally the plant polyphenol is a flavonoid, and preferably a flavonol.

36. The method according to claim 35, wherein the plant polyphenol is selected from hesperidin, quercetin, myricetin, kaempferol, quercitrin, catechin, epigallocatechin gallate, genistein, maringenin, apigenin, luteolin, malvidin, cyanidin and daidzein, or a combination thereof.

37. The method according to claim 33, wherein the tight junction modulator is selected from
i) cis-urocanic acid (cis-UCA), or
ii) ultraviolet irradiated trans-UCA, or
iii) a cis-UCA analogue, optionally selected from 2-pyrroleacrylic acid, 2-thiopheneacrylic acid, 2-turanacrylic acid, dihydrourocanic acid, 2-methylurocanic acid and 3-thiopheneacrylic acid,
or a combination thereof.

38-42. (canceled)
